(19) United States
(12) Patent Application Publication
(10) Pub. No.: US 2008/0041722 A1
(43) Pub. Date: Feb. 21, 2008

(54) METHOD AND DEVICE FOR CONTROLLING ELECTOOSMOTIC FLOW BY USING LIGHT

(76) Inventors: Pal Ormos, Szeged (HU); Andras Der, Szeged (HU); Laszlo Oroszi, Szeged (HU)

Correspondence Address: HAHN & VOIGHT PLLC, 1012 14TH STREET, NW, SUITE 620, WASHINGTON, DC, 20005, US

(21) Appl. No.: 11/875,164

(22) Filed: Oct. 19, 2007

Related U.S. Application Data
(63) Continuation-in-part of application No. PCT/HU2006/000033, filed on Apr. 24, 2006.

(30) Foreign Application Priority Data
Apr. 22, 2005 (HU) P0500406

(51) Int. Cl. C07K 1/26

(52) U.S. Cl. 204/451

(57) ABSTRACT

Electroosmosis is an effective way to move fluids in microfluidic devices. The process is driven by the interaction of a double-layer formed at the surface of the fluid in a microspace of the device and the electric field parallel with the surface. Surface conductivity of light sensitive surfaces can be modified by light and thereby the flow pattern of the fluid can be manipulated. The invention relates to a microfluidic device and method to move fluids by electroosmosis and to manipulate flow of the fluid, wherein at least a part the surface adjacent to the fluid driven by electroosmosis is made of a light sensitive material, the surface conductivity of which is modified by changing illumination thereof, thereby flow of the fluid can be influenced. The invention can be used for example in microfluidic channels to manipulate flow velocity, for the preparation of microfluidic switches and mixers or “lab-on-a-chip” devices.

This is a continuation-in-part of International Application PCT/HU2006/000033, filed Apr. 24, 2006, the entire specification of which is hereby incorporated by reference herein. This application claims priority to Hungarian application P0500406, filed Apr. 22, 2005, the entire specification of which is also incorporated by reference herein.

The invention relates to a microfluidic device and method for manipulating flow properties of a fluid driven by electroosmosis.

The invention can be used first of all in the field of processes and devices suitable for carrying out microscale chemical reactions and chemical and analytical methods. Development in this technical field brought forth a number of miniature devices in which the reacted or studied fluid flows in microchannels (typical cross section: 1 mm>x>10 μm). The device may comprise, besides a microchannel, other miniature functional elements, e.g. a valve or a detector. A detailed review of such devices is given in BioTechniques, Vol. 38, No. 3 (2005), 429-443, together with respective methods of manufacture.

A basic problem of microfluidic devices resides in the provision of a fluid flow of appropriate velocity and direction. A number of solutions of the problem is known; those applied most often are briefly reviewed below.

a) Flow by mechanical pumping. The pump may be located outside the microfluidic device (in this case it is connected to the microfluidic device externally) or may be a part of the device (e.g. the device may comprise a membrane by which pressure can be transmitted to the fluid causing flow thereof).

b) The flow of the fluid is affected by an external electric field (see patent applications US 2002/166595 and US 2003/127329).

c) The fluid flow is driven by centrifugal force which is developed by turning the device around an axis.

d) The fluid flow is driven by magnetic force (for example ferromagnetic particles are dispersed in the fluid).

e) The fluid moves due to capillary effect.

f) The streaming of the fluid is effected by electroosmotic force. Since the present invention provides a significant technical development in the field of electroosmotic fluid handling, below this segment of the art is described in more detail.

Electroosmosis is an efficient and often used method to pump fluids in microfluidic devices. It is based on the phenomenon that if the surface of the wall of a microchannel involving a fluid carries an electric charge (and this is the case in general), then these surface charges must be neutralised locally by the charges in the fluid. At the interface of the oppositely charged fluid and wall, consequently an electric double layer is formed. (From the aspect of practical applications primarily the so called Guy-Chapman layer has to be considered; the so called Stern layer, which is formed by charges tightly attached to the surface, may only modify its effective charge.) The thickness of the double layer is characterised by the so called Debye length (λ_{D}); in the case of aquous solutions it is typically in the order of 10-100 nm (FIG. 1).

The liquid part of the double layer (the charge of which is opposite to that of the channel wall) is displaced by an electric field, whose direction is parallel to the channel wall. If the fluid surface-volume ratio is sufficiently large—and this is the case in microfluidics-, then the thin fluid layer moved by the electric field conveys the full fluid volume with itself, due to internal friction. This process is called electroosmosis. Since the driving force to move the fluid is formed close to the channel wall, the velocity profile characteristic to the fluid flow is different from the parabolic profile of a flow driven by pressure difference; instead, the velocity profile is a flat or closely flat “plug profile”. Due to this feature, and due to the ease of realisation, electroosmosis is often used in microfluidics. The geometry requirements of electroosmosis determine the thickness of the fluid layer, and consequently also that of the microfluidic system.

It is to be noted, that

a) In microfluidic systems the characteristics of fluid flow differ from those in macroscopic systems. In these systems laminar flow is typical, and consequently it is not easy to induce e.g. mixing.

b) Flow velocity is primarily determined by the ionic concentration of the fluid, the electric charge of the channel wall and the magnitude of the electric field effecting the fluid flow.

c) The practical parameters are such that about 100 V/cm electric field is needed to induce a flow velocity of about 100 μm/s.

Since the invention represents a significant practical development in the fluid transport by electroosmosis, this field of the art is described in more detail below.

In the procedure according to the invention, the fluid flow characteristics are manipulated by the application of electromagnetic irradiation in the infrared, visible and ultraviolet spectral range (i.e. visible and non visible light). Therefore, below we shortly overview a few applications where light acts upon the flowing medium in microfluidic devices.

1. There are solutions wherein light acts upon particles floating in the liquid. Upon the effect of light, the particles floating in the liquid will change their properties such that their motion can be modified by an external field (see the International applications WO 02/069016 and WO 02/44689).

2. In the International application WO 2004/012848 a solution is disclosed wherein motion of fluid droplets can be influenced in microfluidic devices. According to this solution the wetting properties of the surface adjacent to the liquid is modified by light, thereby inducing a gradient of the surface tension, and this results in the displacement of the fluid droplets.

The Japanese patent JP 2005-62029 is based on a similar principle (patent application P2003-293531); here the wetting properties of the surface or the hydrophobe-hydrophil properties of the surface are modified by light. The droplets are moved by a method different from electroosmosis.

3. The solution described in patent application US 2002/023840 discloses a manufacturing method, wherein the surface properties of the microfluidic channels are modified by light, and this determines fluid flow in the microfluidic channel. The aforementioned US patent application discloses the production of microfluidic channels, wherein in certain parts of the channel surface (preferably at the outside arc of curvature) the surface charge is modified, for example through immobilizing certain chemicals. This is achieved by laser light illumination. The change of the surface charge on certain areas results in altered flow characteristics compared to other areas of the channel. By this procedure it can be achieved for example that, even in a curve, the plug flow profile is maintained. It must be emphasized that in US 2002/023840 the modification of the surface charge is achieved not during the normal operation of the device or during fluid flow. Moreover, the modifications caused by light are irreversible, i.e. the changes caused by light illumination will persist even after irradiation. Consequently, the role of light is not the control of fluid flow but the induction of a chemical reaction to change the surface properties, i.e. it is used as a tool during the manufacturing method.

4. Moorthy J et al. [Sensors and Actuators B 75 (2001) 223-229] disclose the idea of manipulating electroosmotic flow in a microfluidic device wherein wall of a microchannel is fabricated from a material, more closely TiO₂, the surface charge of which changes upon radiation with UV light. The authors have found that due to the pH dependence of surface charge their method is highly sensitive for pH of the fluid in the microchannel. In particular, among the three pH values tested (pH 2.2, 4.7 and 8.4) only pH 4.7 resulted in a satisfactory mean bulk flow velocity difference between test and control samples, whereas at pH 2.2 and at pH 8.4 the prefix of the surface charge and consequently the direction of flow was quite the opposite. This strong pH dependence clearly indicates that the measured effect is due to a change in surface charge density. Thus, only buffers with stable and well defined pH value can be used at a given pH, which largely limits the practical applicability of the method.

To the best of our knowledge no reliable solution is known in the art for controlling properties of electroosmotic fluid flow in microfluidic devices by light.

The present inventors recognized that if the microchannel wall is covered by a light sensitive material, then by changing intensity and/or wavelength of an illuminating light, the physical state of the said material at the surface adjacent to the fluid and thereby the velocity and/or direction of the fluid flow may be affected or controlled. As a matter of course, it must be ensured that this effect of the light may be achieved during fluid flow. Preferably, the effect of the light is reversible, and after the original illumination level is restored, essentially the original flow properties return again.

Thus, the present invention relates to a method for manipulating flow properties of a fluid driven by electroosmosis in a microfluidic device, comprising the steps of
using a microfluidic device having at least one microspace (microvessel) in which a fluid can be driven by electroosmosis
   and at least a part of the surface of the microspace, said surface being adjacent to of the electroosmotically driven fluid, is made of a light sensitive material,
   wherein the surface conductivity of said light sensitive material changes due to a modification of the illumination, preferably in a reversible manner,
driving a fluid in the microspace by electroosmosis,
modifying illumination of at least a part of the surface adjacent to the driven fluid,
thereby changing the surface conductivity of at least a part of the surface,
whereby flow properties of the fluid in contact with the surface are manipulated.

Preferably, the illumination of at least a part of the surface adjacent to the fluid, is modified one or more times, wherein said surface is made of a light sensitive material.

Preferably, by modifying the illumination the magnitude of the flow velocity of the fluid 14 is increased or decreased.

According to a further, preferred embodiment the direction of the flow velocity of the fluid 14 is modified by modification of the illumination. Optionally, direction and velocity are modified simultaneously.

The microspace of the device is preferably a microchannel 15 wherein the velocity of the fluid 14 driven by electroosmosis is modified by illuminating the photosensitive layer 12 applied to a support 13 by illuminating light 20, or by ceasing illumination thereof.

In the method of the invention preferably the photosensitive material is a photoconductive material, more preferably a CdS containing material is used.

The invention also relates to a microfluidic device useful for driving fluids by electroosmosis and for manipulating flow properties of the driven fluid, said device having a microspace (microvessel) for containing the fluid and, if desired, means for driving the fluid by electroosmosis, wherein at least a part of the surface of the microspace, said surface being adjacent to of the electroosmotically driven fluid, is made of a light sensitive material, the surface conductivity of which changes due to a modification of the illumination, preferably in a reversible manner.

Preferably, the microspace, which allows a fluid to be driven, is formed by one or more microchannel 15.

According to a preferred embodiment the light sensitive material, of which at least a part of the surface adjacent to the fluid is made, is a photoconductive material, preferably a material comprising CdS, optionally it is CdS.

If the light sensitive material is a photoconductive material, according to a preferred embodiment by illuminating at least a part of the surface adjacent to the driven fluid or by increasing intensity of the illuminating light resistance of the photoconductive material may be decreased, whereby in turn the flow velocity of the fluid being in contact with the illuminated surface can be decreased. Preferably, by ceasing illumination or by decreasing the intensity of the illuminating light the flow velocity will increase again.

According to a further embodiment by modifying the illumination the light sensitive material is changed.

In a preferred device the surface adjacent to the fluid 14 driven in the microchannel 15 is composed of the wall 11 and the light sensitive layer 12 applied to a support 13.

Thee device may contain, as means for driving the fluid by electroosmosis, at least one chamber 16 and a reservoir 17 connected thereto, being suitable for containing the fluid 14, and electrodes 18.

According to a preferred embodiment of the device the light sensitive part of the surface adjacent to the fluid is
shaped according to a predetermined pattern or
illuminated according to a predetermined pattern.

Preferably, the device according to the invention comprises a microchannel 15, wherein the light sensitive material is a light sensitive layer 12 and at least a part of the pattern comprises an angled stripe making an angle between 0° and 90°, preferably between 10° and 80°, e.g. between 20° or 30° and 70° or 60°, with the longitudinal axis of the channel, wherein preferably the stripe is formed by

a light sensitive layer, or

a discontinuity in a light sensitive layer or

a surface region having a surface conductivity modified by illumination of a light sensitive layer,

whereby the device is suitable for mixing fluid 14 by illumination of the light sensitive layer 12.

Highly preferably, the light sensitive layer 12, being adjacent to the fluid, is a photoconductive layer 22 applied to a support 13 made of glass, and the stripe is formed by a discontinuity therein.

According to a preferred embodiment the direction of the fluid flow is modified by modifying the illumination of a surface part manufactured according to a predetermined pattern.

According to a further preferred embodiment upon modification of the illumination the light sensitive material is illuminated according to a given pattern.

The invention further relates to a method wherein, in a device according to the invention, the illumination of at least a part of the light sensitive layer 12 on the surface region shaped or illuminated according to a predetermined pattern, thereby modifying the direction of the flow velocity of the fluid 14 driven by electroosmosis, whereby mixing of the fluid is achieved.

According to a further version of the method, in an appropriate device the illumination of at least a part of the surface made of a light sensitive material is modified according to a predetermined pattern, said surface being adjacent to the electroosmotically driven fluid, thereby modifying the direction of the flow velocity of the fluid 14 driven by electroosmosis, whereby mixing of the fluid is achieved.

According to a further preferred embodiment said device comprises more than one microchannels 15 on a support 13 having a two dimensional surface and optionally one or more of the microchannels are connected.

For example, said device comprises the following microchannels 15 on support covered by a light sensitive layer 8, said microchannels 15 being connected by junction 4, in an Y-branch geometry: a main branch 7, a first side branch 5 and a second side branch 6 and, to allow electroosmotic flow of the fluid, in connection with the main branch 7 an at least one first chamber 1 containing the fluid 14.

According to a further preferred method the flow velocity of the fluid 14 is decreased by illuminating at least a part of a first side branch 5, thereby directing the fluid 14 into a second side branch 6, or vica versa, whereby switching is achieved.

The device of the invention may be present in the form of a chip.

The invention also relates to an integrated microfluidic device of the “lab-on-a-chip” type, wherein said integrated microfluidic device comprises more than one devices according to any of claims 7 to 17.

Preferably, the fluid 14 driven by electroosmosis is water or aquous solution.

Highly preferably, the illuminating light is infrared, visible or ultraviolet light depending on the light sensitive material applied.

Some of the terms used in the specification are explained below.

Microfluidic Device

A “microfluidic device” is a miniature device for the realisation of chemical reactions and chemical and biological analytical methods in small sizes, in which the studied or reacting fluid is flowing in a microspace (or microvessel) preferably in thin layer microspaces (typical fluid thickness x is 1 mm>x>10 μm) or microchannels (typical diameter 1 mm>x>10 μm). In the thin layer microvessels the fluid flows between two surfaces, realising a fluid flow geometry in two dimensions. The length of the limiting surface in any direction is much longer than the layer thickness, preferably by at least 10 times. A microchannel is preferably a capillary. In a preferable embodiment the cross section of the channel is of an oblong or closely oblong shape, or of a circle, a circular segment or substantially a circle or a circular segment shape. The simplest embodiment of the device is considered to be a single microchannel, in which we study the fluid flow. A more complex embodiment is a device which comprises several microchannels.

A microfluidic device, in addition to the microchannel, may contain additional miniature functional parts, for example valves, pumps, or detectors. A detailed description of such devices can be found for example in the article BioTechniques, Vol. 38, No 3 (2005) 429-443, together with the description of the methods of production. The teaching of this article is regarded as obviously known for a person skilled in the art.

A preferred embodiment of microfluidic devices are the minute “lab-on-a-chip” type microfluidic devices. Such a device may comprise several functional element (e.g. one or more microchannel/s/, reaction vessel/s/, pump/s/, valve/s/ or detector/s/). Devices of the “lab-on-a-chip” type may be disposable devices for single use only (generally made of plastic), or may be used several times; in the latter case they are often made of glass.

Microfluidic devices may be connected to form a network useful for solving a certain chemical or biological task.

Capillary

A “capillary” is a microchannel, in connection with a given fluid, the phenomenon of capillarity arises or, under given conditions, may arise.

Fluid

The fluid used in the invention may be of any material which is compatible with the material of the microfluidic device and is capable of forming an electrically charged double layer. Preferably aquous solutions are used in which preferably ionic solutes are present.

Fluid Driven by Electroosmosis

In the microfluidic devices of the invention the fluid is driven, at least in part, by electroosmotic pumping. Provided that electroosmotic force contributes, at least partly, to the velocity of a fluid flowing in a capillary or in a microfluidic device, the fluid is considered as driven by electroosmosis. Preferably, the flow velocity of the fluid is determined decisively by the electroosmotic effect.

Manipulating Flow Properties

“Manipulating flow properties” is meant as modifying or altering the magnitude and/or the direction of the flow velocity of a fluid driven by electroosmosis in a microfluidic device. In the simplest case the fluid flow is slowed down or accelerated. However, if the light sensitive superficial material is illuminated and an appropriate geometry is provided the direction of the flow can be modified as well.

Surface Adjacent to a Fluid

A “surface adjacent to a fluid” is meant herein as the surface of a microspace or microchannel being in contact with the flowing fluid. In practice the surface is a thin layer of finite thickness and its material property defines the electroosmotic flow under the given physical conditions. According to the invention a material property of at least a part of the surface adjacent to a fluid may change due to a modification in the illumination. Such a surface, forming boundary of a fluid and having a property changeable by the action of light, may be prepared e.g. by creating a coating, preferably a thin layer, of a light sensitive material.

Light

The term “light” in the specification covers both visible and non-visible light, e.g. preferably includes ultraviolet, visible and infrared light. For practical applications light within the 400 to 700 nm wavelength (i.e. substantially the visible) range appears to be advantageous. It is to be noted that use of monochromatic light (e.g. laser) may often be preferred. The light to be used depends of course from the light sensitive material applied.

Modification of Illumination

According to the invention flow properties may be manipulated, e.g. controlled or switched by light. Upon modification of illumination, we modify the illumination of that part of the surface adjacent to the fluid, which is covered by light sensitive material. More closely, modification of illumination is understood as a modification in the intensity and/or wavelength of the light effecting a change in the surface conductivity (or specific resistivity) of the light sensitive material at the surface. A change of these material properties then results in the manipulation of the flow. A modification of illumination involves the case when the light causing the change of property, is e.g. switched off (dark state).

Reversible change of a property is a change which partly or entirely disappears after the effect causing it has been ceased. Preferably, the property is restored at least in 50%, preferably the original property is substantially restored.

Light Sensitive Material

A “light sensitive material” is a material the surface charge, surface charge density or surface conductivity (or resistivity) of which changes due to a modification of illumination (in short: by the action of light). In the microfluidic devices of the invention at least a part of the microchannel wall is either made of a light sensitive material or coated by such a material, preferably by a thin layer thereof.

If the charge density of a surface is changed by the action of light, this results in a change of the charge density of the fluid layer being in contact with the surface of altered charge density. This, in turn, results in an alteration of fluid flow raised by the electric field. Such a light sensitive material is, for example but not limited thereto, TiO₂, SnO₂, WO₃, V₂O₅; these materials emit electrons by the action of light; a feature contributing to changing the surface charge. Such materials may be e.g. films of certain proteins, e.g. bacteriorhodopsin. The present inventors found that if essentially only the surface charge of a material, e.g. TiO₂, is changed upon illumination, the effect on flow velocity is rather small and it is inappropriate for practical purposes.

If the surface conductivity is changed by the action of light, this results in the change of the electric field strength in the fluid being in contact with the surface; thereby the flow of the fluid due to the electric field will be modified. If the resistivity of the surface is decreased, preferably decreases to at least 60% or 50%, preferably 40%, 30% or even 20% or 10% of the original resistivity, preferably the surface becomes conductive or more conductive, the material is considered photoconductive (or photoelectric, i.e. becoming conductive by the action of light). As used herein, a material is “conductive” if it is able to conduct electricity, e.g., a conductor or a semiconductor.

In this case the reduced resistivity decreases the electric field driving the electroosmotic flow.

The photoconductive phenomenon may occur e.g. in semiconductors. The phenomenon is facilitated by defects in crystals (lattice disorders) or contaminations. Materials like these are oxides, sulphides or selenides etc. of certain metals, e.g. thallium, titanium, cadmium or lead, etc. Upon experimentation the present inventors found that as a light sensitive material can be selected for example from the following: an amorphous Si layer, light sensitive metal-oxide and/or metal-sulphide layers, preferably a CdS layer. (The surface conductivity of the CdS layer is increased by the action of light.)

For a given light sensitive material a person skilled in the art will readily determine the appropriate intensity and wavelength of the light used to illuminate the light sensitive surface, so that the desired change in its material properties and the subsequent modification of fluid flow may occur.

Surface Conductivity and Surface Charge Density

In a broader sense surface conductivity is meant as the conductivity of a given surface layer, defined or measured by any known way.

For example, in a typical case the surface conductivity as a material property can be calculated as follows:

If, on a given surface, potential difference U raises current I between electrodes (or material parts serving as electrodes for the given surface) of width D, wherein the distance between the said is L, then the surface resistivity is

$${ρ}_{f} = \frac{\frac{U}{L}}{\frac{I}{D}} \qquad ( I )$$

Surface conductivity is the reciprocal of surface resistivity.

Surface charge density is defined by the number of charges on a given surface in a surface layer, for example if the quantity of charges is Q and the surface area is A than the surface charge density is given as
σ=Q/A. (II)

### BRIEF DESCRIPTION OF THE FIGURES

FIG. 1 shows schematically the structure of the double layer evolving near to the surface adjacent to the fluid.

FIG. 2.A shows the cross-section of a channel used in the experiments to demonstrate the phenomenon of photoelectroosmosis.

FIG. 2.B shows the longitudinal section of a simple, experimental microfluidic device. The device comprises a single microchannel and means for driving the fluid therein.

In FIG. 3.A an experimental arrangement is shown useful for studying, by measurements, the exemplary microfluidic devices of the invention.

FIG. 3.B shows the screen for a computer analysis of a fluid driven in a microchannel of 200 μm width. On the left side of the figure identified and tracked polystyrene beads can be observed. At the upper right part of the screen the parameters of identification and tracking can be set. Dots seen at the lower right part give the velocity vectors.

In FIG. 3.C manipulation of the fluid flow velocity by light is illustrated. In the figure the average flow velocity values are indicated in alternating dark an illuminated states. As a coating CdS was used.

FIG. 4 shows a pattern created in a light sensitive layer on the wall (here actually the bottom) of a linear microchannel. This pattern is suitable to achieve mixing even in microfluidic devices providing laminar flow. It can be seen that there is a stripe which is actually formed by a break or discontinuity of the light sensitive material.

In FIG. 5 different flow patterns observed in the dark (FIG. 5.A) and on illumination (FIG. 5.B) are shown, in a microchannel comprising on its wall the pattern suitable to achieve mixing, as shown on FIG. 4. Analysis was carried out by observing the polystyrene beads added to the flowing fluid and, so as to illustrate and visualize the flow, the photo images of the beads were blurred along the direction of the motion.

FIG. 6.A a flow pattern (velocity distribution) is shown in the dark state, in a microchannel suitable for mixing. The analysis was made by the program illustrated on FIG. 3.B. One dot represents the velocity of a polystyrene bead.

In FIG. 6.B the velocity distribution of the beads is shown upon illumination, otherwise in the same experimental arrangement as in FIG. 6.A.

The simulated flow pattern in FIG. 7 was prepared by the FEMLAB program to show the results of the same mixing method illustrated on FIGS. 4 to 6.

In FIG. 8.A a schematic top-view drawing of a light controllable microfluidic switch of the Y-branch type is shown, in a state without illumination.

In FIG. 8.B the same Y-branch type switch as in FIG. 8.A is shown in the illuminated state.

FIG. 9 represents the flow in the Y-junction upon illumination. The simulation was carried out by the FEMLAB computer program.

FIG. 10.A is a photo of a simple microfluidic device comprising an Y-branch type switch and useful for studying the said switch type. The length of the linear segments is 5-5 mm each, the distance between the two side branches of the Y is 6 mm.

FIG. 10.B is a photo of the Y-junction in the switch shown on FIG. 10.A. The photo was made during operation of the device, without illumination thereof. It is apparent that the polystyrene beads are present in both branches.

FIG. 11.A shows the velocity distribution of the polystyrene beads in the switch of Y-branch geometry, without illumination. The results were obtained by the computer analysis illustrated in FIG. 3.B. One dot represents the velocity of a single bead.

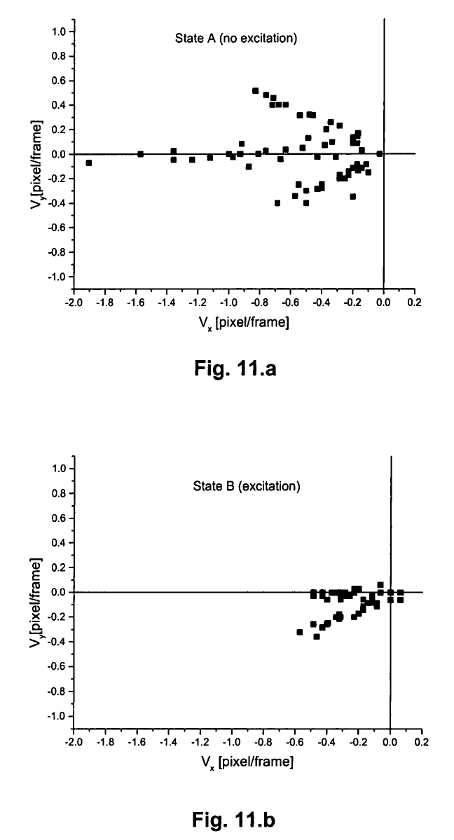

In FIG. 11.A a velocity distribution characteristic to the same switch is shown, if one of the side branches of the Y-branch switch is illuminated as illustrated in FIG. 8.B.

### DETAILED DESCRIPTION OF THE INVENTION

Below the invention is described in more detail. The examples given below are merely illustrative.

Upon manufacture of the microfluidic device of the invention one or more microspace (microvessel) is created. Depending on geometry, the microspace may be e.g. a channel, if its length significantly exceeds its width and height (hereafter: microchannel), or may be e.g. a thin layer if its length and width significantly exceeds its height, i.e. theoretically the fluid therein may move into in two dimensions. It is to be noted that the terms length, width and height are merely relative terms to describe the shape of the microspace and do not indicate the position of the microspace or the device compared with a direction defined outside the device (e.g. horizontal).

The cross section of the microchannel may be of any shape, preferably an ellipse, e.g. a circle, or a quadrangle, e.g. a rectangle (oblong).

So as to create the microspaces of the device preferably a photolithographic method is applied. The primary material of the device may be glass or, preferably, plastic, e.g. silicone rubber, preferably poly(dimethyl-siloxane) (PDMS). Various materials may also be used together. The cross section of the microchannel depends on the manufacturing technology. For example, if a photolithographic method is used, it will be of oblong shape the typical size of which is between 10 and 1000 μm, preferably between 50 to 500 μm. Other plastics which are often used are TPE (thermoset polyester), PMMA (poly-methyl-methacrylate), PC (polycarbonate), COC (cyclic olefin copolymer), PS (polystyrene), PVC (polyvinyl-chloride), or PETG (polyethylene terephthalate glycol). A number of microfluidic devices of various types are described by Fiorini, G. S. and Chiu D. T. [BioTechniques Vol 38, No 3 (2005) 429-443] which, together with methods for preparing microfluidic devices cited therein, is incorporated herein by reference. As to the material of the wall of the microspace in the device it is a requirement that between the fluid and the wall an electronic double layer may be formed and thereby the fluid may be driven by electroosmosis. Various materials which fulfil this requirement are well-known by a skilled person.

As explained above, a basic feature of the microfluidic device of the invention is that at least a part of the surface being in contact with the fluid driven by electroosmosis is covered by a light sensitive material. By the action of light a certain property of the surface covered by a light sensitive material changes; this change affects the electroosmotic force driving the fluid and consequently the flow properties of the fluid are modified.

The surface property changing due to the action of light is surface conductivity (or resistivity). It is to be noted that in practice in certain cases both surface charge density and surface conductivity may contribute to the desired effect. However, it is found by the inventors that a change in surface conductivity is essential whereas a change in surface charge density appears to be insufficient in itself to provide a practically useful effect. Usually, even in such cases one of the effects (a change in a material property) dominates over the other: in general, these cases are referred to herein based on the dominant effect.

The light sensitive material may be used to create the microchannel wall (or bottom) therefrom or it may be applied, e.g. as a thin layer, to the wall or bottom of the microspace. The advantage of the latter method is that illumination may be carried out from either side by the illuminating light. The thickness of the layer preferably is in the nm to μm range. In the example described herein a CdS layer of 90 nm was applied.

According to the invention any material can be used as a light sensitive material which shows the desired change in its property by the action of light. Expediently, the change of the property is reversible. As an example for materials changing their surface charge by the action of light TiO₂ may be mentioned. Materials changing their surface conductivity by the action of light may be sought among amorphous semiconductor materials. Such materials are e.g. a Si layer, As, Se or sulphide layers, light sensitive metal-oxides and/or metal-sulphides, preferably CdS (the surface conductivity of the latter increases by the action of light).

It is to be emphasized that a number of photoconductive materials is known according to the art. A few examples are described herein, and as examples, devices and methods are provided herein useful for studying these materials. A simulation method is also described to demonstrate that applicability of these materials can be predicted to a certain extent by simulation. Having this information the skilled person will be able to test further light sensitive materials and define whether they are applicable according to the invention, without difficulty; moreover, optimizing the parameters of the application is also within his/her ability.

Once a given photoconductive material is selected, the wavelength (or wavelength range) as well as the intensity of the light (e.g. infrared, visible or ultraviolet light) to be applied is either immediately apparent or can be defined by simple experimentation. Thus, the inventive idea lies at a first place not in the definition of these material properties; these are merely examples which are not intended to define or limit the claimed scope.

An advantageous feature of the invention is that by modifying the intensity of the illuminating light the electroosmotic force can be either increased or decreased gradually (within the range allowed by the given parameters), preferably in a reversible manner, and thereby the fluid flow may be accelerated or slowed down, or the direction of the velocity vector may be changed thereby achieving mixing.

Either the area, the number or the pattern of the light sensitive surfaces may be varied according to the invention. As a matter of course, within a single device it is possible to create more than one light sensitive surfaces which are not connected to each other. The illuminated surface, either its area or its shape, can also be varied as well as the time of illumination. Light sensitive layers from more than one type of material may be applied, allowing for example the control of the fluid flow by multiple kinds of light.

Various patterns suitable to influence the fluid flow properties may be created according to the invention. For example, the light sensitive layer may be applied according to a given pattern. In this case the pattern is formed by light sensitive and non light sensitive surface parts. The shapes of the pattern elements may be diverse, like a stripe, a bow or bend, it may have a break or a corner. A simple solution, useful to modify flow direction besides flow speed is to apply a stripe the direction of which makes an angle of more than 0° and less than 90° with the main flow direction, e.g. with the longitudinal axis of the channel. The stripe may be of light sensitive material whereas the rest of the surface is not light sensitive, or the stripe may be a break or discontinuity in larger, light sensitive surface. If within a light sensitive surface regions (“holes”) are provided, which are not light sensitive, this is also considered a discontinuity.

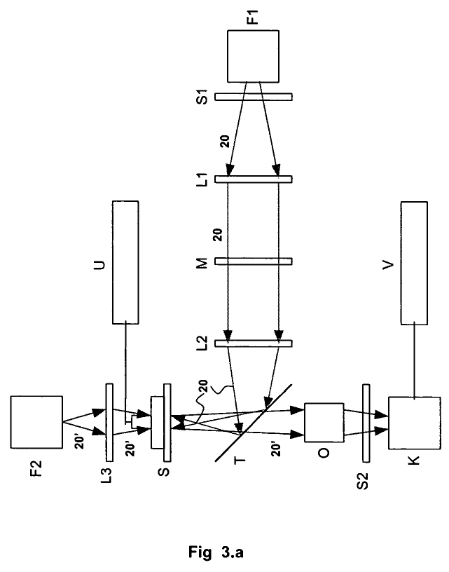

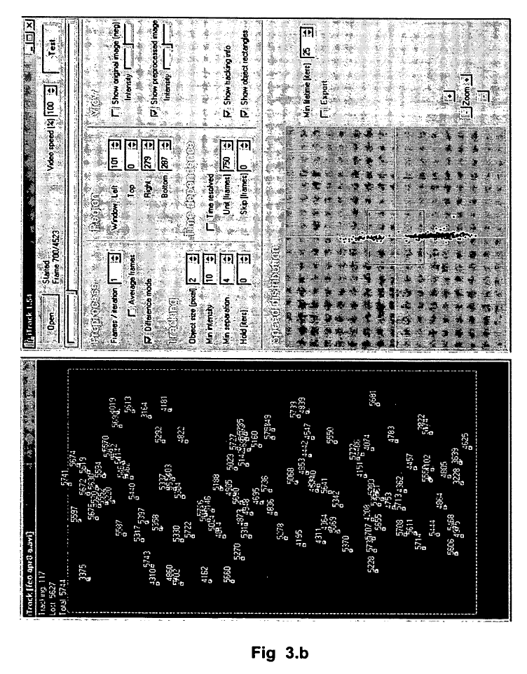

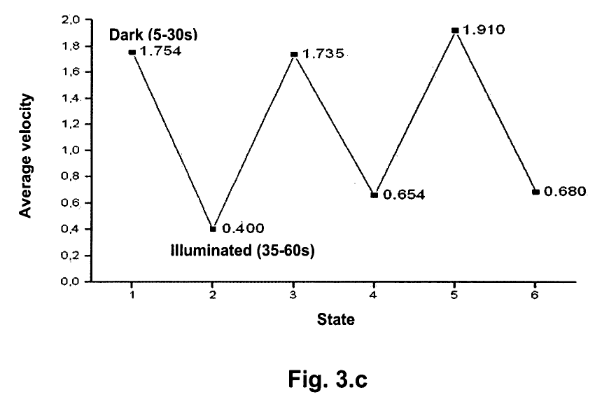

The same type of patterns may be created by projecting these patterns onto the light sensitive surface according to the invention, e.g. by the experimental setup shown on FIG. 3.A. The advantage of this embodiment is that the pattern is freely changeable; thereby a variety of flow profiles may be created by the same microfluidic device.

According to a further embodiment the pattern is provided by the combination of a multiplicity of light sensitive materials. According to a preferred version these are sensitive to different light wavelengths.

Expediently, these steps are controlled by computer. The variations listed above permit to carry out even very complicated fluid manipulation methods. The exemplary simulation program and method allows simulation of these manipulation methods and prediction of the effect of various patterns. The analysis method taught herein facilitates testing of various patterns or appropriate design of the devices.

In the examples described herein the light sensitive layer was applied to only one of the walls of the of the channel (its “bottom”); nevertheless, this provided a sufficient result as to the modification of the flow pattern. It is evident that in an appropriate size-scale (determination of which is well within the abilities of a person skilled in the art) either one or more walls of the microspace or the microchannel may be covered by the light sensitive layer. Thereby the effect may be increased or more complicated patterns may be created.

According to the invention in one device more than one microspace, e.g. microchannel may be assembled, or even a complex system may be built. The microchannels may be connected; in this case the fluid flow may be slowed down or blocked in any of them thereby arbitrarily directing the fluid into any of the microchannels. In other words, the fluid flow may be switched among the microchannels.

According to the invention the fluid is water or aquous solution. As a matter of course, partly or entirely, a fluid made of other solvent may also be used, provided that a double layer along the surface adjacent to said liquid may be formed allowing driving the fluid by electroosmosis. Theory and practice of electroosmosis is well-known in the art. Moreover, capillary zone electrophoresis is based on electroosmosis [c.f. Gáspár A.: “Kapilláris zónaelektroforézis, Egyetemi Kiadó, Debrecen, Hungary, (2000)], which, besides its many practical applications, is a widely used method to move fluids in microspaces (microvessels). For years of development of capillary zone electrophoresis a huge amount of experience has been accumulated. Based on this knowledge determination of the composition of the fluid (solvent, ionic strength, solved components) and adaptation of the method of the invention to a given task is far from being difficult; moreover, the skilled person will also be able to select an appropriate material of the wall of microspace in respect of a given fluid.

As a matter of course, development of many technical details may require the creation of further dependent solution within the scope of the present invention, provided that these solutions are based on the present inventive idea.

Below a few exemplary applications of the present invention are discussed.

By altering the velocity of fluid flow the duration of a given step of a process may be controlled. Moreover, this can be achieved separately in each microchannel, which allows a complex regulation on a single chip.

Fast fluid flow is not always advantageous in microfluidics. For example, slower fluid flow permits sometimes a shorter capillary length or facilitates better separation, e.g. by capillary electrophoresis.

Efficiency of separation may be improved by periodically altering the flow velocity, which may result in a better isolation of particles of different motility, from each other.

If a chemical reaction takes place in the capillary, by the method of the invention the flow velocity of the fluid may be adjusted to the reaction rate.

As in microfluids the flow is essentially always laminar, by slowing down the flow, mixing of components by diffusion occurs, if it is “waited out”.

Mixing can be effected by creating a given flow profile. For example, a helical flow pattern can be achieved which facilitates mixing. A flow pattern inducing mixing may also be created by illuminating a surface of appropriate pattern or by applying the light sensitive material in an appropriate pattern then illuminating it.

The plug flow profile may be maintained even in a curve or in a turn of the capillary if only one side of its wall is illuminated. For example, at the inner side of the curve the flow may be slowed down. (This embodiment may resemble to the solution taught in US2002/023840 with the fundamental difference that in the present invention illumination is applied during fluid flow, preferably in a reversible manner.)

A number of possible applications may be based on a combination of electroosmotic and traditional pumping. For example, by applying an electroosmotic flow perpendicular to the direction of mechanical pumping two dimensional separation may be achieved. In this case the fluid should preferably flow in a thin layer. By applying a simultaneous illumination the particles to be separated may be brought out at different ports.

A light controlled version of the microfluidic “bump array” may be carried out as well. Bump array is a microfluid particle separating tool which comprises physical obstacles to control fluid flow. Based on this principle a system can be designed in which at certain carefully chosen points (bumps) the fluid flow differs from that at other parts of the surface. A difference for example in flow direction may result in the formation of local flow cells. A change in the distribution of the “bumps” permits separation of particles of different size.

According to the invention a device can also be made in which the fluid by be transferred from a main branch to any of one or more side branches, and the fluid flow may be switched among the side branches as explained above. The principle may be used for separation of fluids of different composition, e.g. for fraction collection or for selection of suspended particles, e.g. cells, microspheres etc.

One or more of the elements disclosed above, e.g. microchannels, microlayers, mixers, switches, branches may be integrated on a chip, thereby creating “lab-on-a-chip” type devices.

In the Examples below a few preferred examples are taught merely for the purpose of illustration. A skilled person will be able, of course, to carry out the invention defined in the claims in a variety of modified embodiments.

### EXAMPLES

### Example 1

### Manufacture and Operation of a Simple Experimental Microfluidic Device

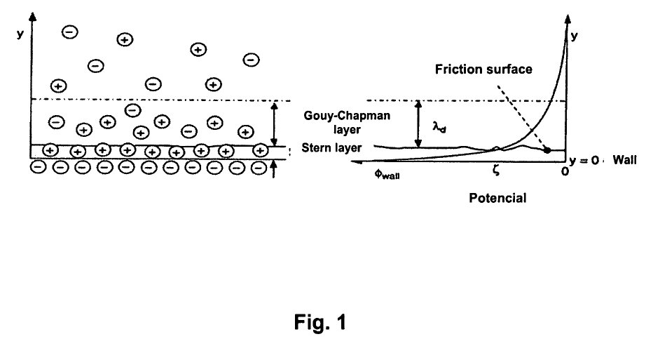

A microfluidic channel of simple geometry was created by the procedure described below. The cross section of the microchannel 15 is shown in FIG. 1.B.

A chromium mask of the channel was prepared by photolythographic procedure (Mikro T Kft, HU, www.microt.hu). The negative version of the structure was made on a glass surface by the method of photopolymerization by photolythography with the negative tone photoresist Michrochem Nano SU8. The photoexposure of the chromium mask was achieved in the usual way, the light effecting solidification was provided by a Teiss HBO100 Mercury lamp. Subsequently, the final channel was molded with this negative form with the silicone rubber PDMS (Sylguard 184 Silicone Elastomer, Dow Corning Chemicals). The process can be carried out based on the information provided by the suppliers or manufacturers. PDMS was found to be highly preferable because it is an inert material and solidifies from a liquid. In our example the PDMS mold is placed upon a glass support 13 and so the microchannel 15 is formed, the wall 11 of which is formed by the PDMS mold. In our experiments we used channels with characteristic sizes of 200 μm width and 100 μm height.

For the preparation of the device according to the invention the support 13 was covered by the light sensitive layer 12, e.g. by any of the methods described in example 3.

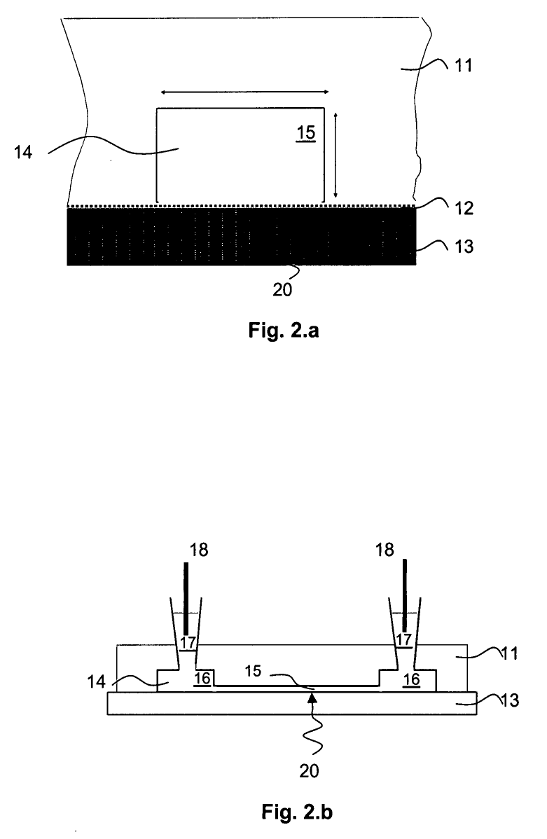

The vertical section of the full microfluidic device is shown in FIG. 2.B. In the device chambers 16 and reservoirs 17 were formed. The chambers 16 and cavities incorporating the reservoirs 17 were also produced by the microlithographic procedure described above, however, these can be prepared with any body with the proper shape, and subsequently producing the PDMS mold using the form. The device was subsequently filled up with fluid 14′.

The early experiments were performed with channels of 100 μm height, 200 μm width and 1 cm length. The electroosmotic flow of fluid 14 in the microchannel 15 was induced using electrodes 18 made of platinum (Pt). In this case the electrolyte was distilled water, or a salt of low ionic concentration. The voltage (30-150V) applied upon the Pt electrodes 18 was provided by a power supply (see example 2.2.) During the operation of the device, while fluid 14 flows from one chamber 16 into the other chamber 16, the fluid 14′ in one of the reservoirs 17 is depleted while the other reservoir 17 is filled. In a simple version of the device the direction of flow is determined only by the polarity of the applied voltage.

The flow velocity is influenced by the illuminating light 20, thereby controlling or switching it. Namely, with the illuminating light 20 we modify a surface property of a surface adjacent to the fluid, e.g. the light sensitive layer 12 covering support 13. More closely its surface conductivity (see e.g. CdS, Example 3.1.) may be changed. This modifies the state of the electrical double layer determining the electroosmotic flow on the channel bottom (see FIG. 1) that in turn alters the velocity of electroosmotic flow of fluid 14. If the electric field component pointing in the direction of the flow is decreased (e.g. because the surface conductivity of the light sensitive material 12 is increasing) the flow velocity will decrease. If the polarity of the double layer is increasing (e.g. the surface charge of the light sensitive material is increasing) the flow velocity will increase, too.

### Example 2

### 2.1 Experimental Arrangement to Study Devices of the Invention

When the invention was reduced into practice we prepared several basic microfluidic devices (simple microchannel in which the fluid flow velocity can be manipulated, Y-branch design channel junction, microfluidic mixer). To test these devices we built up the experimental arrangement shown in FIG. 3.A. The experimental arrangement consists of two main parts, one of which contains the means for the operation of the microfluidics system. The other part was used for the characterization of the fluid flow (see Example 2.2.).

Among the means needed for the operation the electric voltage, providing the driving force (30-150V), was supplied by the power supply U (PSE300 direct current power supply, Biocenter Kft). Illuminating light 20 controlling the illumination of light sensitive layer of sample S was provided by illuminating light source F1 (in the experiments with CdS this was a mercury lamp HBO50). To determine the shape of the illuminated area the projected pattern M (e.g. slit, grating) was projected upon the light sensitive layer of sample S by using illuminating light 20 from illuminating light source F1 passing through the bandpass filter S1 (400-600 nm) and application of lenses L1 and L2.

In order to study fluid flow particles used to visualize flow (in the given example we mixed small polystyrene beads to the fluid, see Example 2.2.) were observed with the camera K and the images were recorded with video recorder V. Illumination of the sample was achieved with light 20′ of the LED light source F2 (753 nm wavelength). Imaging of the sample upon the sensor of the camera was achieved with a long working distance objective O (M Plan Apo, 10X, /0.28, Mitutoyo). The filter S2 placed in front of the camera prevented that undesired light (ambient light, light of the illuminated pattern) reach the camera. The beam splitter mirror T reflected the light of the illuminating light source F1 onto the sample, and permitted the light 20′ from LED F2 to reach the camera.

### 2.2. Method to Characterize Fluid Flow We followed fluid flow in a microscope, and visualized it by mixing polystyrene beads of 1 μm diameter into the fluid. Motion was followed by a TV camera, the films were recorded by a computer (movie file) and they were analyzed by a motion analyzer computer program.

As an example, FIG. 3.B. shows the analysis of flow in a channel of 200 μm width. On the left side of the figure an image of the channel is shown with the identified beads just tracked. In the upper right part the parameters in connection with identification and tracking of the beads are set. The point in the lower right part of the figure represent the determined velocity vectors.

Characterization of the flow. Individual tracking of the particles.

Each single moving particle was identified, tracked and its motion was characterized. The behaviour of the system was given by the statistics of these individual motion data. The image analysis program identifies the individual moving particles, tracks them and then to the full motion a velocity value is assigned (from the moment it was found, until the moment it was finally lost). (For reliable operation of the program the parameters of the image analysis program had to be set independently for each film—after a certain practice this is a fairly fast process.) The statistics of these velocity values provides the characterization of the system.

The input of the program is a movie file. In the operational mode used in the present analysis the program subtracts (pixel by pixel) the subsequent images of the movie file from each other (‘Difference mode’ parameter); consequently, only those features are seen that move. Furthermore, there is a possibility to average subsequent images and the differences of these averages can be used for analysis (parameters ‘Frames/iteration’ and ‘Average frames’). This feature may be of advantage in the case of noisy films, or if the flow is very slow and consequently the difference image has not sufficient contrast. The program searches for particles (detection) and tracking is performed upon the sequence of this set of preprocessed images.

Detection is achieved the following way: for each pixel the average intensity is calculated in a point's neighborhood having a given size (object size), and if this average intensity value is larger than a threshold value (min intensity—characteristic to each film and set by the user), than the given point is accepted as a hit. During tracking of a particle in the subsequent film frames the point pairs are identified. We regard a newly detected object ‘A’ identical to object ‘B’ in the previous image if ‘A’ is the closest to ‘B’ among all of the images identified in the previous frame and likewise, ‘B’ is closest to ‘A’ among the newly detected objects.

It could happen that to a single spot several objects would be assigned, since within a single spot the conditions of detection could be fulfilled in several pixels. These undesired cases can be excluded by setting a minimum object distance value (min. separation). (The parameter ‘hold’ has no role in this version. This parameter enables the user to re-identify a lost particle: its value sets the number of frames for which the particle is not regarded as lost—e.g. if it is behind an other object for a period, etc—thereby providing continuity to the observation.)

The output of the program is a set of the velocity vectors of the followed objects (velocity distribution). A velocity vector is calculated from the distance of the positions where the particle was found (emerging at the edge of the picture) and lost (disappearance at the opposite edge of the picture) and the time between these events. [During the presentation of the velocity distribution and export of the data it is possible to filter the data based on a minimal duration of tracking (min lifetime)].

Other settings of the program serve to set the active investigated area in the film, where tracking is performed (‘Region’), and divide the film into time periods, within which we wish to determine the velocity distribution independently from other time periods in order to find a time dependence of these values (‘Time dependence’).

The parameter values seen in the display image of the program represent typical values used in our experiments.

The program was written in the development system C++ Builder 4.0 in the Institute of Biophysics, in object oriented concept.

### 2.3. Computer Simulation of the Investigated Processes

With the program FEMLAB (Comsol FEMLAB 3 Multiphysics Modeling) we carried out the full computer simulation of the studied processes. This is very important, since the system and the studied processes are very complex and consequently it is impossible to predict the behaviour intuitively.

In the first step of the simulation the geometry of the system to be modelled is defined. Then we calculate the spatial structure of the electric field in the application mode Conductive Media DC (electrostatics of conductive media). As a next step in the “Incompressible Navier-Stokes Steady-State analysis” mode we determined the fluid flow in such a way that the effect of electroosmosis was taken into account as a boundary condition based on the Helmholz-Smoluchowski relation. The parameters of the different materials building up the system (e.g. conductivities, viscosities) and the boundary conditions (e.g. insulators, continuity, electric potential) were set so that they correspond to realistic parameters of the experiments or literature data.

We used the computer simulation to model the microfluidic mixing and the switch of Y design, in order to make sure that we understand and are able to simulate the complicated effects influenced by electrostatics, electroosmotics and hydrodynamics. The results clearly show that modeling is a very useful method in the further development of the devices of the invention or in the development of more complex lab-on-a-chip devices.

### Example 3

### Examination of Light Sensitive CdS Layer

### Control of Fluid Flow Velocity

The CdS layer represents one type of light sensitive layers in the concept of the invention, the materials that change their electrical conductance upon light illumination.

The glass support of the microchannel produced according to example 1 was covered with a CdS layer of about 90 nm thickness by the method of chemical deposition [Chemical bath deposition of CdS and CdPbS nanocrystalline thin films and investigation of their photoconductivity, Mater. Res. Soc. Symp. Proc. Vol. 900E© 2006 Materials Research Society].

The experiments were performed in the simple device disclosed in FIGS. 2.A. and 2.B., in microchannels 15 of 100 μm height, 200 μm width and 1 cm length. The electric field was applied to the sample with Pt electrodes 18. The fluid was distilled water or water solution of low ionic strength. The sample was illuminated with illuminating light 20 of spectral region 400-600 nm from a mercury-vapor lamp.

The results obtained are shown in FIG. 3.C. In the figure the change of fluid flow upon light illumination can be seen. In the experiment light and dark periods were following each other. On the effect of illumination (light period) the flow velocity was reduced to a fraction of the original, and when the illumination was stopped (dark period) the original flow velocity was recovered. The basis of the change is that the decrease of the resistance on the channel base (bottom) reduces the electric field that drives the electroosmotic flow.

### Example 4

### Mixing by Light

In the case of microfluidic devices it is particularly important to achieve efficient mixing, since due to the low Reynolds-number the flow is always laminar, and mixing occurs only through diffusion and consequently it can be very slow.

### 4.1. Creating of a Steady Pattern in a Light Sensitive Layer

In this experiment the mixing was carried out in a way that allows to introduce a steady pattern into the layer, and the activity was controlled by light activation.

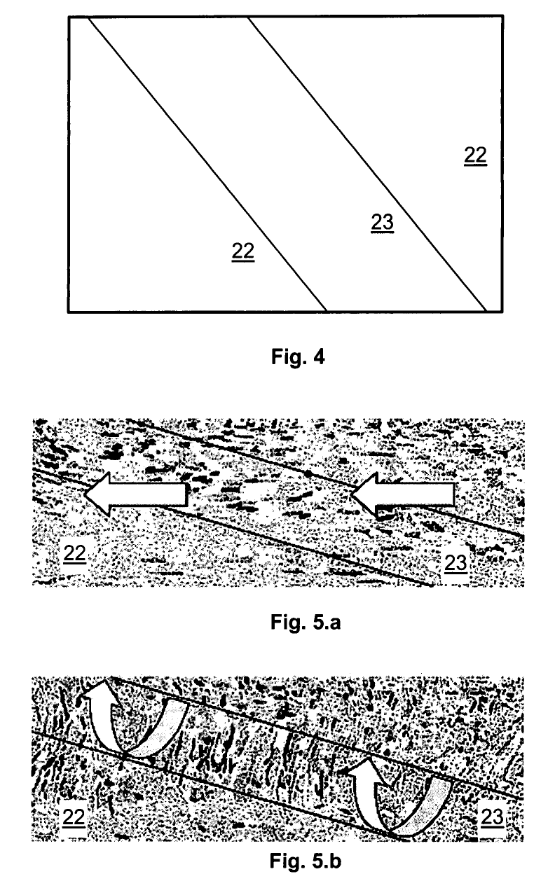

The pattern which was introduced into the photoconductor on the bottom of the channel is shown in FIG. 4. The continuity of the photoconductive layer 22 (in this example the CdS layer) was broken by an angled stripe (making an angle between 0 and 90° with the axis of the channel), thereby the stripe 23 was formed. The material of stripe 23 corresponding to the material of the support 13 was glass, and its width was 100 μm. In every other aspect the channel was identical to that described above. The flow observed in the dark and under illumination is shown in the FIGS. 5.A. and 5.B.

In the dark, when the resistance of the CdS layer is large, the flow is simple linear, just as in the ground case (FIG. 5.A.). However, upon illumination the flow pattern changes dramatically: The average velocity is decreased and along a cylinder over the stripe 23 a circular flow pattern evolved (FIG. 5.B.). We illustrated the flow by blurring the photo images of the beads along the direction of the motion. The quantitative analysis was carried out here, too, by tracking the small beads mixed to the fluid.

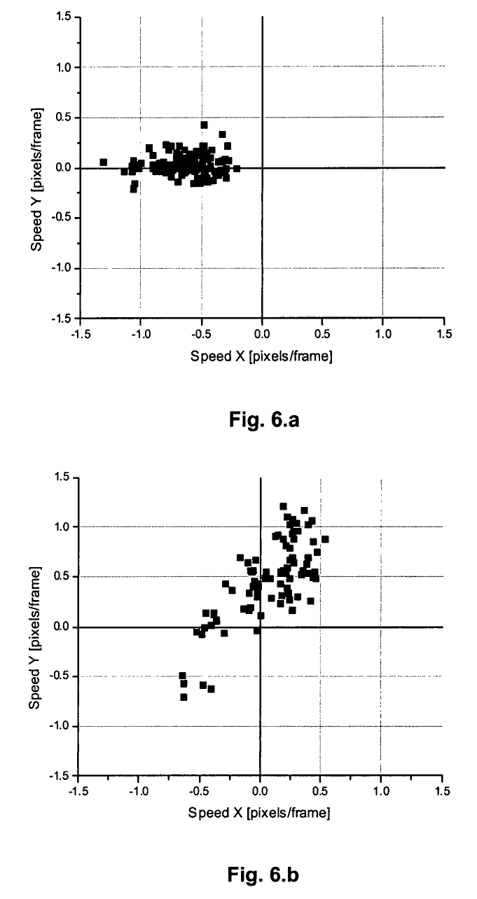

The motion of the beads was analyzed with the program described in example 2.1. In FIG. 6.A. the velocity distribution of the beads in the channel is shown when there is no illumination. In the figure one dot represents to the x and y components of the velocity, i.e. it represents the horizontal components, parallel to the support. In the case without illumination we see a simple flow profile: the velocities point in one direction and they are practically equal.

During illumination, however, the velocity directions are perpendicular to the direction of the glass stripe and they point to both directions (FIG. 6.B.). The image analysis program does not distinguish the motions on the top and on the bottom, consequently, this distribution shows a cylindrical flow. In the figure the number of velocity vectors pointing up and down or left and right are not equal. The reason for this is that the different motions take place in different heights, and these are found by the program with unequal probability. The centre of an imaginary oval shape covering the dots representing the velocities can be found left from the origin. This shows that there is an average flow to the left, as expected, corresponding to the main flow direction.

We simulated the phenomenon with the procedure explained above, with realistic parameter values, and the obtained image is shown in FIG. 7. It is apparent that the simulation yields basically the same pattern as the experiments. The successful simulation is a proof that the formation of the flow pattern was understood.

We can conclude that we solved the mixing of fluid in microfluidic channels. This can be switched on and off by light. We are convinced that this will be an important element of microfluidic devices. For example, we can accelerate a diffusion limited second order chemical reaction. This may have exceptional significance in the case of the analysis of DNA-chips, where detection time could be significantly reduced.

### 4.2. Pattern Generation by Illumination, Changing the Flow Direction by Light

The pattern of the change of conductivity, and so the helical flow can also be realized by projecting the appropriate pattern upon the surface, i.e. the parameters of mixing can also be adjusted by light.

The experiment is the following: In the experimental arrangement described in example 1 one side of the channel is covered by a light sensitive material (FIG. 2.A.) Applying an illumination with intensity distribution of appropriate pattern, we can modify the direction of flow. If we illuminate the surface with stripes making an angle with the channel direction smaller than direct angle, then it will conduct flow along the light sensitive surface along these stripes, in analogy with the case described in example 4.1.

The mixing realized in microfluidic channels solves an old problem, and it enables uniform distribution of solvents or the development of e.g. microreactors.

### Example 5

### Optically Controlled Microfluidic Y-Branch Switch

In the simple linear channel described in Examples 1 and 3 we could change fluid flow velocity by illumination by about an order of magnitude. We use this phenomenon in the example shown in FIGS. 8.A and 8.B to select and control, by illuminating light 20, the direction of fluid flow in such a way that in the selected side-branch of the Y junction 4 (e.g. first side-branch 5 FIG. 8.B.) flow is stopped by illumination, thereby the flow is directed into the other side-branch (second side-branch 6). The switch was manufactured as disclosed below.

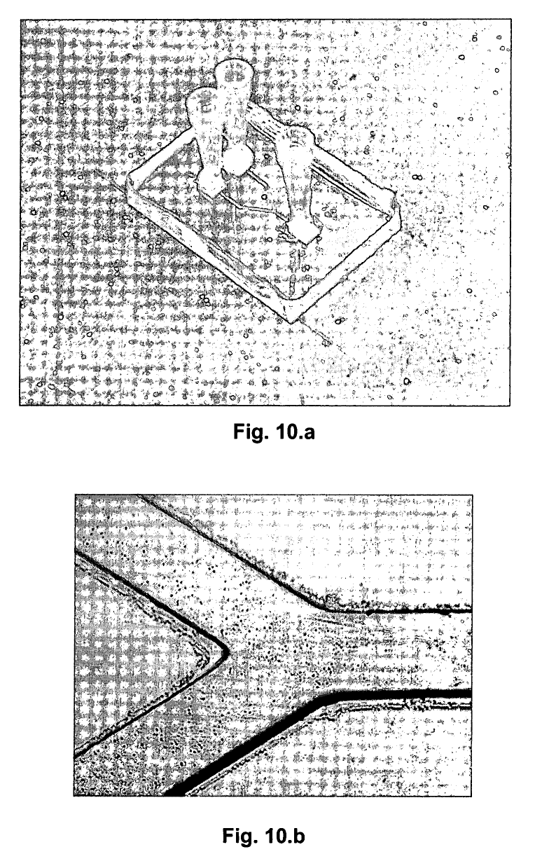

By the procedure described in Example 1 we produced a channel of Y-branch geometry. The light sensitive layer 12 in this example was CdS. The channel used in the experiments and shown on the photograph of FIG. 10.A, had the following parameters: each straight section was 5 mm long, the distance between the two side branches of the Y-branch is 6 mm, the area of the chambers: 3×3 mm. The rest of the parameters were identical to those given in Example 1.

The chambers 1 and 2 are on identical electrical potential. In the dark, flow starts from chamber 3 and it goes into chamber 1 and chamber 2 in equal distribution. FIG. 8.A. shows the flow without illumination, while FIG. 8.B. shows the device under illumination. The illuminated area is marked by the circle. If, behind junction 4, one side of the channel is illuminated, no fluid will flow into that channel, so the fluid will flow only into chamber 2 (the arrows represent the flow direction in the figure).

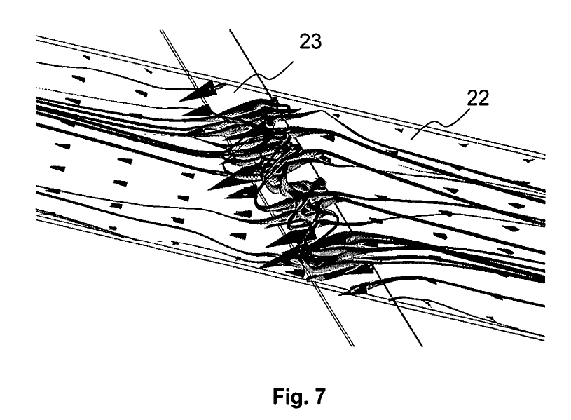

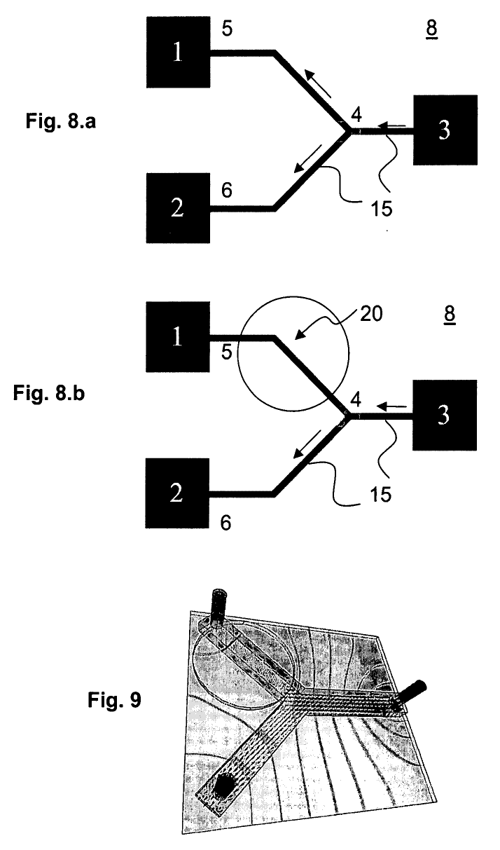

We performed computer modeling of the processes in the Y-branch the results of which are shown in FIG. 9. The laws of hydrodynamics, electrostatics and electroosmosis were taken into account with appropriate parameters.

In the figure the surfaces represented by ribbons denote equipotential surfaces. The sample is illuminated in the marked area, wherein the resistance of the surface decreases. It can be seen that over the surface the electric field is expelled from the channel. The fluid velocity is represented in each location by the small cones; the heights of the cones correspond to the velocity values in that particular point. It is apparent that illumination stops the flow in the upper, illuminated side-branch.

We emphasize that simulation was performed with realistic parameters.

In FIG. 10.B. we show a photograph of the vicinity of junction 4. We visualize fluid flow exactly as earlier: we mix microscopic polystyrene beads into the fluid, we follow them, and characterize their motion by computer motion analysis. In the photograph the beads are seen as small black dots.

The flow is represented as the velocity distribution of the beads as in example 2.2.

The result of the analysis is shown in FIGS. 11.A. and 11.B. In the state without illumination represented on FIG. 11.A. the beads travel both in the upper and lower branches with equal distribution. Subsequently, the Y-branch channel is illuminated as seen in FIG. 11.B. Now the fluid flow occurs only in the second side channel 6 (bottom) into second chamber 2 (see also FIG. 8.B.). There is no fluid flow into the third chamber 3. Observation was done at junction 4, and the area observed contained proportional parts of the main branch as well as that of the two side branches.

With the above device we carried out the optical switching of fluid flow. This switching element may become an important component of microfluidic systems. Based on the principle of the Y-branch, e.g. a microfluidic cell sorter can be constructed, that could be completely controlled by light. In one embodiment of the device in the main branch 7 we detect a certain parameter of the cells. For example, if a certain fluorescent dye is present in the cell, the fluorescent signal can be detected (this is a common procedure in cell cytometry) The signal controls whether side branch 5 or 6 has to be illuminated, and so fluid flow is directed into one or the other branch, thereby effectively selecting the cells.

Based on the same principle, fraction collection can also be performed in a microfluidic device. If during the separation procedure, e.g. a capillary electrophoretic procedure or microchromatographic procedure, the material to be separated appears in the effluent, and this event is detected by a detector attached to the main branch, using the signal so obtained the illumination of the side branches can be controlled. For example, as long as only buffer is flowing out from the separation device, it is directed into the first side branch 5 by illuminating side branch 6 and it is discharged. As soon as the detector attached to main branch 7 detects that the material to be separated flows through the microchannel, side branch 5 is illuminated and the desired material is collected in the second chamber 2. By choosing the threshold level of the detector the purity of the fraction can be determined.

### Example 6

Comparison of the Photoconductive Properties of CdS and Titanium-Oxide Layers

Surface Resistivity of CdS and TiO₂ Layers.

All layers have been obtained from the Institute of Solid State Physics and Optics of the Hungarian Academy of Sciences (Budapest, Hungary). CdS was prepared by chemical deposition, the titanium oxide layers were prepared evaporation of a mixture of titanium oxides of the approx. stochiometry of Ti₄O₇. In case of TiO₂ evaporation was carried out under oxidative conditions. All layers had a thickness between 100 and 250 nm.

The layers were illuminated with light of 12 mW/cm² intensity (this saturated the light effect in CdS) as described in Example 3. Electrodes were made of conductive epoxy material. Both width of electrodes and their distance were approx. 1 cm. The layers were illuminated both from the side of the glass support and from the other side to prevent any disturbing effect of the glass. However, results did not vary upon the illumination side.

| Material | Illumination | Surface resistivity (MOhm) |
|---|---|---|
| CdS | Dark | 19.0 |
| | Light | 5.2 |
| CdS-2 | Dark | 9.0 |
| | Light | 2.2 |
| Titanium oxide | Dark | 0.2 |
| (approx. | Light | 0.198 (barely detectable) |
| stoch.: Ti₄O₇) | | |
| TiO₂ | Dark | >100 MOhms (unmeasurable) |
| | Light | >100 MOhms (unmeasurable) |
| TiO₂ | Dark | >100 MOhms (unmeasurable) |
| | Light | >100 MOhms (unmeasurable) |

The resistivity of titanium oxide did not change practically upon illumination. The resistivity of TiO₂ oxidised was too high to measure any alteration, but the actual value is irrelevant, because at this high resistance values it is definitely not applicable for modulating the electric field in microfluidics channel by photoconductivity.

Titanium oxide comprises a mixture of a number of oxides wherein the oxidation degree of titanium is varied. As a result, its resistivity is rather low. It is known, however, that TiO₂ comprises small radius polarons, which though are capable of provide electrons upon illumination, these electrons do not move freely. As an explanation it can be reasonably assumed that if these polarons are near to the surface, they contribute to a surface polarity change, but do not result in an increase in conductivity.

Quite to the contrary, the conductivity of CdS is significantly increased upon illumination.

Consequently, it can be generally stated that Titanium oxide layers, e.g. TiO₂ can not be used to modulate electroosmosis by its photoconductivity. Thus titanium oxides or TiO₂ are inappropriate for use in the present invention.

### REFERENCE NUMBERS

M projected pattern
F1 illuminating light source
S1 bandpass filter
L1, L2 lenses
S sample
K camera
V video recorder
F2 LED
O long working distance objective
S2 filter
T beam splitter mirror
U power supply
1 first chamber
2 second chamber
3 third chamber
4 junction
5 first side branch
6 second side branch
7 main branch
8 support covered by light sensitive layer
11 wall
12 light sensitive layer
13 support
14, 14′ fluid
15 microchannel
16 chamber
17 reservoir
18 electrode
20 illuminating light
20′ light
22 photoconductive layer
23 stripe

1. A method for manipulating flow properties of a fluid driven by electroosmosis in a microfluidic device, characterized by
using a microfluidic device having at least one microspace which is suitable for a fluid to be driven by electroosmosis wherein at least a part of the surface of the microspace, being adjacent to the electroosmotically driven fluid, is made of a light sensitive material the surface conductivity of which changes, preferably in a reversible manner, due to a modification of the illumination,
driving a fluid in the microspace by electroosmosis,
modifying illumination of at least a part of the surface adjacent to the driven fluid, thereby
changing the surface conductivity of at least a part of the surface, whereby
flow properties of the fluid in contact with the surface are manipulated.

2. The method according to claim 1, characterized by modifying one or more times the illumination of at least a part of the surface adjacent to the fluid, wherein said surface is made of a photoconductive material.

3. The method according to claim 1 characterized by manipulating, e.g. increasing or decreasing the magnitude of the flow velocity of the fluid 14 by modification of the illumination.

4. The method according to claim 1 characterized by manipulating the direction of the flow velocity of the fluid 14 by modification of the illumination.

5. The method of claim 1 characterized in that the microspace of the device is a microchannel 15 wherein the velocity of the fluid 14 driven by electroosmosis is modified by illuminating the photosensitive layer 12 applied to a support 13 by illuminating light 20, or by ceasing illumination thereof.

6. The method of claim 2 characterized in that the photoconductive material is an amorphous semiconductor material or a metal oxide, sulphide or selenide, e.g. CdS.

7. A microfluidic device useful for driving fluids by electroosmosis and for manipulating flow properties of the driven fluid, said device having a microspace for containing the fluid and, if desired, means for driving the fluid by electroosmosis, characterized in that
at least a part of the surface of the microspace, said surface being adjacent to the electroosmotically driven fluid, is made of a light sensitive material the surface conductivity of which changes, preferably in a reversible manner, due to a modification of the illumination.

8. The device of claim 7 characterized in that the microspace, which allows a fluid to be driven, is formed by one or more microchannel 15.

9. The device of claim 7 characterized in that the light sensitive material of which at least a part of the surface adjacent to the fluid is made is a photoconductive material, preferably a photoconductive material comprising an amorphous semiconductor material or a metal oxide, sulphide or selenide, preferably CdS, optionally the photoconductive material is CdS.

10. The device of claim 7 characterized in that the light sensitive material is a material which is different from a TiO₂ layer changing its surface conductivity by the action of light.

11. The device of claim 8 characterized in that the surface forming adjacent to the fluid 14 driven in the microchannel 15 is composed of the wall 11 and the light sensitive layer 12 applied to a support 13.

12. The device of claim 11 characterized in that, as means for driving the fluid by electroosmosis, said device comprises at least one chamber 16 and a reservoir 17 connected thereto, being suitable for containing the fluid 14, and electrodes 18.

13. The device according to claim 7, characterized in that the light sensitive part of the surface adjacent to the fluid is
shaped according to a predetermined pattern or
illuminated according to a predetermined pattern.

14. The device according to claim 13 comprising a microchannel 15 for driving the fluid 14, wherein the light sensitive material is a light sensitive layer 12 and at least a part of the pattern comprises an angled stripe making an angle between 0° and 90°, preferably between 10° and 80°, e.g. between 20° or 30° and 70° or 60°, with the longitudinal axis of the channel, wherein preferably the stripe is formed by
a light sensitive layer, or
a discontinuity in a light sensitive layer or
a surface region having a surface conductivity modified by illumination of a light sensitive layer,
whereby the device is suitable for mixing fluid 14 by illumination of the light sensitive layer 12.

15. The device according to claim 14 characterized in that the light sensitive layer 12, said layer being adjacent to the fluid, is a photoconductive layer 22 applied to a support 13 made of glass, and the stripe is formed by a discontinuity therein.

16. The microfluidic device of claim 7, characterized in that said device comprises more than one microchannels 15 on a support 13 having a two dimensional surface and optionally on or more of the microchannels are connected.

17. The microfluidic device of claim 16, characterized in that said device comprises the following microchannels 15 on support covered by a light sensitive layer 8, said microchannels 15 being connected by junction 4, in an Y-branch geometry: a main branch 7, a first side branch 5 and a second side branch 6 and, to allow electroosmotic flow of the fluid, in connection with the main branch 7 an at least one first chamber 1 containing the fluid 14.

18. The method of claim 1 characterized by modifying,
in a microfluidic device useful for driving fluids by electroosmosis and for manipulating flow properties of the driven fluid, said device having a microspace for containing the fluid and, if desired, means for driving the fluid by electroosmosis, characterized in that at least a part of the surface of the microspace, said surface being adjacent to the electroosmotically driven fluid, is made of a light sensitive material the surface conductivity of which changes, preferably in a reversible manner, due to a modification of the illumination, said device further characterized in that the light sensitive part of the surface adjacent to the fluid is shaped according to a predetermined pattern or illuminated according to a predetermined pattern,
the illumination of at least a part of the light sensitive layer 12 on the surface region shaped or illuminated according to a predetermined pattern, thereby manipulating the direction of the flow velocity of the fluid 14 driven by electroosmosis, whereby mixing of the fluid is achieved.

19. The method of claim 1 characterized by modifying according to a predetermined pattern,
in a microfluidic device useful for driving fluids by electroosmosis and for manipulating flow properties of the driven fluid, said device having a microspace for containing the fluid and, if desired, means for driving the fluid by electroosmosis, characterized in that at least a part of the surface of the microspace, said surface being adjacent to the electroosmotically driven fluid, is made of a light sensitive material the surface conductivity of which changes, preferably in a reversible manner, due to a modification of the illumination,
the illumination of at least a part of the surface made of a light sensitive material, said surface being adjacent to the electroosmotically driven fluid, thereby manipulating the direction of the flow velocity of the fluid 14 driven by electroosmosis, whereby mixing of the fluid is achieved.

20. An integrated microfluidic device of the “lab-on-a-chip” type, characterized in that said integrated microfluidic device comprises more than one devices according to claim 7.
